(19) United States
(12) Patent Application Publication
(10) Pub. No.: US 2025/0351893 A1
(43) Pub. Date: Nov. 20, 2025

(54) Maternity Pants and Maternity Leggings Having an Integral User-Adjustable Belly-Support Band

(71) Applicant: Delta Galil Industries Ltd., Caesarea (IL)

(72) Inventors: Iris Yemini Basson, Hod HaSharon (IL); Andrea Dorazilová, Abu Ghosh (IL); Lea Esther Hay Pardo Harel, Eshhar (IL); Diana Del Gadillo, Nahariya (IL); Shlomit Amar, Haifa (IL)

(21) Appl. No.: 19/108,165

(22) PCT Filed: Sep. 6, 2023
(86) PCT No.: PCT/IL2023/050957

§ 371 Date: Mar. 2, 2025

Related U.S. Application Data
(60) Provisional application No. 63/374,965, filed on Sep. 8, 2022.

(51) Int. Cl. A41C 1/10; A41D 1/21

(52) U.S. Cl. CPC A41C 1/10; A41D 1/21

(57) ABSTRACT

Maternity pants and maternity leggings having an integral user-adjustable belly-support elastic fabric band or elastic fabric strap. The elastic fabric band remains connected to the rear side of the pants or leggings, and can be angularly pivoted or slanted upwardly, to cover an upper region of the pregnant belly of the wearer, while leaving an uncovered front-side aperture that enables a portion of the pregnant belly to remain uncovered. Optionally, a lower-back covering fabric-region of the pants or leggings provides lower-back support, by providing one or two generally-horizontal elastic bands that are generally parallel to the waistband.

### CROSS-REFERENCE TO RELATED APPLICATIONS

This patent application claims priority and benefit from U.S. 63/374,965, filed on Sep. 8, 2022, which is hereby incorporated by reference in its entirety.

### FIELD

Some embodiments are related to the field of clothing.

### BACKGROUND

Clothing articles and clothes are typically formed of textile material and are worn on the body. Clothes are worn for various purposes, for example, to keep the human body warm in a cold weather, to protect the human body from rough surfaces or insects or plants, to provide a hygienic barrier between the human body and the environment, to protect the human body from ultraviolet radiation, to cover genitals, for social reasons or as fashion, or the like.

### SUMMARY

Maternity pants and maternity leggings (which may be long, or short) having an integral user-adjustable belly-support elastic fabric band or elastic fabric strap. For example, the elastic fabric band remains connected to the rear side of the pants or leggings, and can be angularly pivoted or slanted upwardly, to cover an upper region of the pregnant belly of the wearer, while leaving an uncovered front-side aperture that enables a portion of the pregnant belly to remain uncovered. Optionally, a lower-back covering fabric-region of the pants or leggings provides lower-back support, by providing one or two generally-horizontal elastic bands that are generally parallel to the waistband.

Some embodiments provide maternity pants or maternity leggings, that integrally include at least one selectively-adjustable and/or location-modifiable (or slanting modifiable, or angular location modifiable) fabric strap or fabric belt or fabric band or fabric strip, for supporting the belly; the fabric-based belt or band may be selectively moved or adjusted by the wearer, upwardly or downwardly, to be above the navel (belly-button) or below the navel or covering the navel, and to provide comfortable and user-adjustable support according to the particular needs of the wearer and according to the gradual growth of the pregnant belly; and may also provide support to the lower-back region of the wearer.

In some embodiments, the belt or band may me selectively moved and positioned by the wearer, at an upper region or central region or lower region of the pregnant belly, or below the pregnant belly as a cradle support element; or may be angularly placed or positioned to selectively cover only the belly-region that is above the navel; or may be angularly placed or positioned to selectively cover only the belly-region that is beneath the navel; or may be angularly placed or positioned to selectively cover only the belly-region that covers the navel and leaves at least one belly-region uncovered (e.g., above the covered navel region; or below the covered navel region; or both above and below the covered navel region); or may be angularly placed or positioned to selectively cover both the belly-region that covers the navel and the belly-region that is beneath the navel, while leaving uncovered a top or upper belly-region that is above the navel; or may be angularly placed or positioned to selectively cover both the belly-region that covers the navel and the belly-region that is above the navel, while leaving uncovered a bottom or lower belly-region that is beneath the navel; or may be angularly placed or positioned to cover the entirety of the pregnant belly (above the navel, and covering the navel, and beneath the navel). The belly-support region or band or belt is fabric-based, and is formed of a fabric that is knitted or woven from yarn(s).

In some embodiments, at least some of the yarns of the belly-support region band have a first elasticity property; and at least some other of the yarns of the belly-support region band have a second, different, elasticity property.

In some embodiments, a front-side of the belly-support region band has a first elasticity property; and a rear-side of the belly-support region band has a second, different, elasticity property.

In some embodiments, a front-side of the belly-support region band has a first elasticity property; and a rear-side of the belly-support region band has a second, reduced, elasticity property.

In some embodiments, optionally, the belly-support components or belt(s) or band(s) or fabric strip(s) or fabric strap(s) are an integral and non-detachable part of the pants or leggings; thereby forming a combined, unified, singular, hybrid garment, and not requiring the user to maintain and to wear two separate garments (e.g., pants, and a separate support belt). In other embodiments, the belly-support components may be implemented as a stand-alone accessory or garment. In other embodiments, the belly-support components may be detachably attached to pants or leggings (or other garment) via a connection mechanism that allows attachment and detachment (e.g., Velcro, zipper, fasteners, or the like); such that the garment is still a singular or unified or hybrid garment, yet one particular component thereof (namely, the belly-support region) may optionally (in some implementations) be removed by the wearer, if no support at all is desired, or in order to wash or clean only that garment-portion.

In some embodiments, at least a portion of the front side of the pregnant belly remains uncovered; for example, to enable that body-region to be in contact with fresh air and/or to be cooled off by fresh air or by the nearby environment, or to allow release of sweat, or for fashion or aesthetic reasons of the wearer.

Some embodiments may provide other and/or additional benefits and/or advantages.

### BRIEF DESCRIPTION OF THE DRAWINGS

FIG. 1 is an illustration of Maternity Pants/Maternity Leggings, in accordance with some demonstrative embodiments.

FIG. 2 is an illustration demonstrating a single band which may be formed and then cut to produce three garment-components, in accordance with some embodiments.

FIG. 3 is an illustration of a garment-portion in accordance with some demonstrative embodiments.

FIG. 4A is an illustration of pants/leggings and an associated belly-support unit, in accordance with some demonstrative embodiments.

FIG. 4B is an illustration of short pants/short leggings and an associated belly-support unit, in accordance with some demonstrative embodiments.

FIGS. 5A and 5B and 5C are illustrations of some fabric components of the maternity pants/leggings, in accordance with some demonstrative embodiments.

FIG. 6A is an illustration of a belly-support clothing article, in accordance with some demonstrative embodiments.

FIG. 6B is an illustration of a clothing article such as pants/leggings, which integrally comprises a knitted fabric-made belly-support region, in accordance with some demonstrative embodiments.

FIG. 7A is an illustration of a belly-support clothing article, shown schematically as being worn by a pregnant female, in accordance with some demonstrative embodiments.

FIG. 7B is an illustration of a front-side of a portion of a clothing article, in accordance with some demonstrative embodiments.

FIG. 7C is an illustration of a rear-side of a portion of a clothing article, in accordance with some demonstrative embodiments.

FIG. 8 is an illustration of maternity pants/leggings, having an integrated belly-support region that covers at least a portion of the pregnant belly, and also an integrated and movable belly-support strap that can be moved by the human wearer to provide cover and/or support to a particular region of the pregnant belly, in accordance with some demonstrative embodiments.

FIG. 9A is an illustration demonstrating an implementation of a clothing article that provides adjustable/movable front-side belly-support, as well as back-side (e.g., lower back) support, in accordance with some demonstrative embodiments.

FIG. 9B is an illustration demonstrating another implementation of a clothing article that provides adjustable/movable front-side belly-support, as well as back-side (e.g., lower back) support, in accordance with some demonstrative embodiments.

FIGS. 10A and 10B are illustrations of a clothing article having an adjustable and movable front-side belly-support strap, as well as back-side (e.g., lower back) support, in accordance with some demonstrative embodiments.

### DETAILED DESCRIPTION OF SOME DEMONSTRATIVE EMBODIMENTS

The Applicants have realized that some pants or leggings may not be comfortable for pregnant females, and/or may not provide efficient support and/or efficient body coverage to some pregnant females, and/or may not provide adequate or sufficient support and/or body coverage to some pregnant females.

For example, realized the Applicants, conventional pants or leggings may end (at their upper region) at or around the waist area of the wearer; and may not cover and/or support and/or encircle the bottom region of the belly of a pregnant wearer.

Some maternity pants, realized the Applicant, may comprise an upper-side extension circular (or tube shaped), that may enable some pregnant females to pull up that upper-side tube-shape extension; thereby allowing some pregnant females to stretch upwardly the upper region of the pants, to provide partial coverage to the bottom region of the pregnant belly. However, realized the Applicants, such conventional products often cover only the lower region (e.g., the lower third, or the lower half) of the pregnant belly, and do not extend to (and do not cover) the upper region (e.g., the upper third, or the upper half) of the pregnant belly.

Additionally or alternatively, realized the Applicants, some pregnant females may prefer to cover and/or to support, via their maternity pants or maternity leggings, both (I) a lower region (e.g., the lower one-third or the lower one-half) of the pregnant belly, and also (II) an upper region (e.g., the upper one-third or the upper one-quarter or the upper one-fifth) of the pregnant belly, via a single, integrated, clothing article; while also leaving uncovered a middle region (e.g., the middle one-third, or a middle region that is approximately 25 or 35 percent) of the pregnant belly; for example, to allow that middle-region of the pregnant belly to be in contact with fresh air and/or to allow it to release sweat, and/or for aesthetic purposes or fashion purposes (e.g., similar to females who wear a Belly Shirt that leaves a portion of the belly exposed or uncovered).

Additionally or alternatively, realized the Applicants, there is a need to provide to pregnant females an innovative type of maternity pants and maternity leggings, that provide additional and needed support (physical support) to the back or lower-back region of the female wearer, and/or to the lower-region of the pregnant belly, and/or to the upper-region of the pregnant belly; while also allowing the female wearer to leave uncovered and/or exposed at least a portion of a front-side of the pregnant belly (e.g., for fresh air, to release sweat, for aesthetic or fashion reasons, or the like).

The Applicants have further realized that some pregnant women suffer from low back pain or from lower back pain; for example, due to excess belly weight, which puts a strain on the joints of the lower back and/or pelvis; and that innovative pants/leggings may alleviate or reduce such pain, for example, by incorporating detachable or non-detachable support straps or support belts, each such belt extending from the top-rear region of the pants/leggings and circulating forwardly to encircle a region of the pregnant belly. The innovative pants/leggings with multiple Support Belts mechanisms provides an adaptive physical support to the pregnant belly as it increases in size and/or changes in three-dimensional contour, together with physical support to the lower back region of the female wearer; allowing such support during and along all the pregnancy period, as well as post-delivery/post-pregnancy/post-maternity.

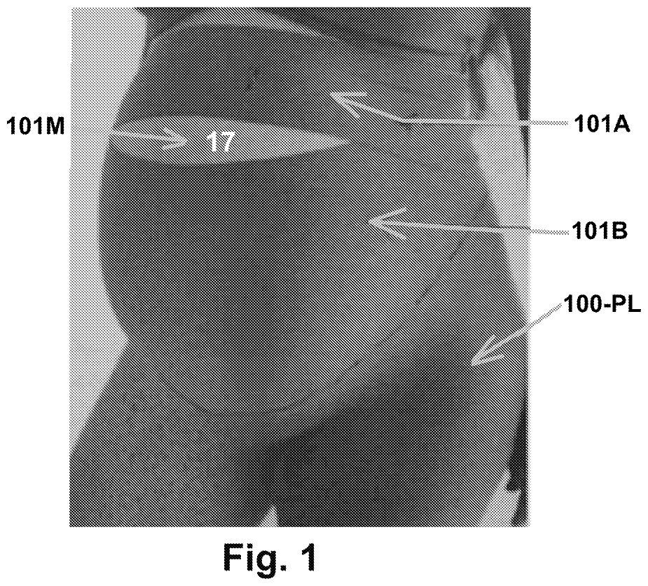

Reference is made to FIG. 1, which is an illustration of Maternity Pants/Maternity Leggings 100-PL (wherein PL stands for Pants/Leggings), in accordance with some demonstrative embodiments. The central or upper fabric-region of the Pants/Leggings (PL) is generally similar to conventional pants or leggings, and/or to maternity pants or maternity leggings, as it covers the legs or hips, the front-side region of genitalia, the crotch/anus, the buttocks, and encircles the waist of the wearer. Fabric-region 101A is an upper support strap or belt made of elastic fabric; Region 101B is a lower support strap or belt made of elastic fabric; Region 101M indicates a middle region or a central region that is exposed or remains uncovered. Fabric Regions 101A and 101B and 100-PL are all integrally knitted together, as a unified, singular, integrated, clothing article. In accordance with some embodiments, fabric region 101A cannot be disconnected or removed from the Maternity Pants/Maternity Leggings 100-PL. In accordance with some embodiments, fabric region 101B cannot be disconnected or removed from the Maternity Pants/Maternity Leggings 100-PL.

In some embodiments, the upper support strap (101A) and the lower support (101B) may be integral and integrated fabric elements, or integrally-knitted fabric-made elements, that are integrally connected or glued or attached to the top-back-side or the top-side or the top-rear side of the Pants/Leggings; and cannot be detached or removed therefrom (without damaging the clothing article). In other embodiments, the upper support strap (A) and the lower support (B) are add-on knitted items, that can be detachably-attached to the top-back-side or the top-side or the top-rear side of the Pants/Leggings, and can be later detached or removed therefrom without damaging the clothing article.

### First Set of Demonstrative Embodiments

In some embodiments, the interactive and adaptive support belts may include three main components: (A) an upper support belt, which provides support to an upper region of the pregnant belly and also to the lower back of the female wearer; (B) a lower support belt, which provides support to a lower region of the pregnant belly and also to the lower back of the female wearer; (C) a stretchable support fabric-region, that is located between or near support belts A and B.

The support belts (A and B) and the stretchable support fabric-region C, may be formed by any suitable technology, for example, knitting, weaving, seamless knitting, or the like. Their production may include plain knitting, rib, pique, mesh, and/or other types of knitting structures or knitting techniques, single-layer, double-layer, or a combination of two or more of the above. They may be formed of cotton yarn(s), polyester yarn(s), nylon yarn(s), nylon 6 yarn(s), nylon 66 or nylon 6/6 yarn(s), modal yarn(s), synthetic yarn(s), semi-synthetic yarn(s), natural yarn(s), yarn(s) from beechwood tree or beech tree, yarn(s) made of wood pulp based cellulosic fiber (e.g., European Schneider Zelkova tree, Chinese Zelkova, Zelkova Schneideriana), rayon fiber yarn(s), viscose fiber yarn(s), Spandex or Elastane or high-elasticity yarn(s) (e.g., able to stretch to at least 3 or 4 or 5 times of its idle length or size), elastic filament yarn(s), elastic spun yarn(s), and/or a combination or mixture of two or more of the above.

In a demonstrative non-limiting example, each of support belts A and B is a double-layer seamless knitted belt or band or strap, formed of a mixture or combination of Nylon and Spandex yarns that are knitted with rib structure, to achieve adequate elongation and recovery of each belt/strap/band, and to achieve soft hand feel and comfortable fit.

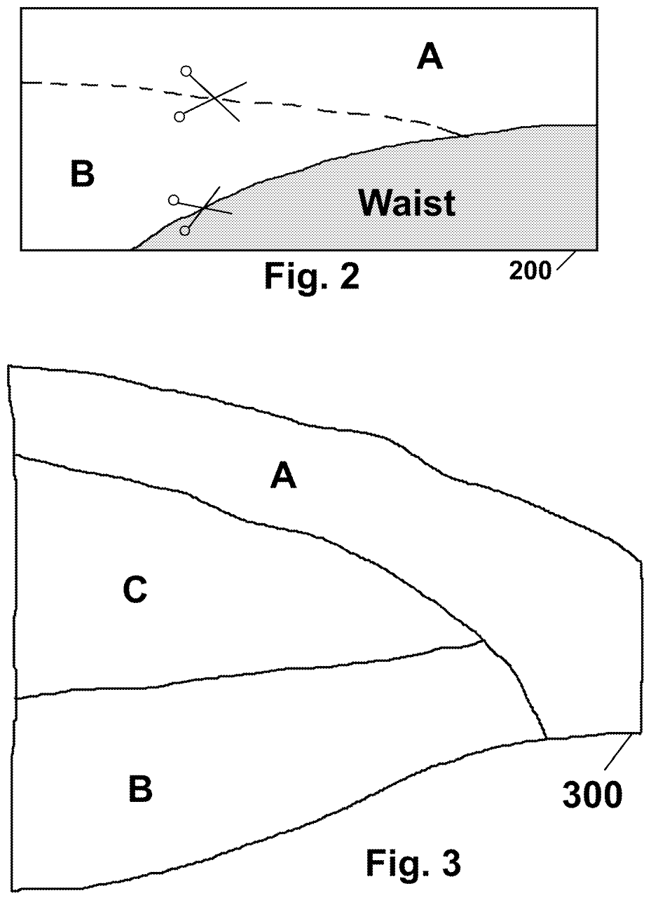

Reference is made to FIG. 2, which is an illustration demonstrating a single band 200 which may be formed and then cut to produce three garment-components, in accordance with some embodiments. In a demonstrative example, a single band 200 is formed, and is then can (e.g., with scissors or a blade or other cutting unit) into three separate bands (belt A, belt B, and a waist region) as shown.

Then, as further shown in FIG. 3, fabric-region C may be produced, as a four-direction stretchable component, which may be formed of Spandex and/or Nylon. The fabric-region C is connected between support belts A and B, for example, by sewing with a folded binding/elastic component, with two needles covered stitch. In some embodiments, the connecting of the three components (A, B, C) may be performed using bonding, gluing, ultrasonic bonding/gluing, a process of bonding/gluing together with ultrasonic vibrations, and/or other connecting mechanisms.

Reference is made to FIG. 3, which is an illustration of a garment-portion 300 in accordance with some demonstrative embodiments. It demonstrates support belt A and support belt B, after their connection to support fabric-region C (e.g., via knitted connection; seamless knitted connection; gluing/bonding connection; ultrasonic bonding/gluing connection).

In some embodiments, the triple-component belly-support system may optionally be implemented as a stand-alone or independent unit or accessory, without necessarily being integrated to (or non-detachably attached to) leggings or pants; such that the pregnant female (or other female, including post-pregnant females or non-pregnant persons) may wear them over or beneath other clothing articles (e.g., skirt, dress, short pants, high-waist briefs, long pants, leggings, underwear). In some embodiments, optionally, a suitable connection mechanism may be used to allow connection or attachment of the stand-alone/independent accessory to such other clothing article/s, for example, via a zipper, a fastener, a hook-and-eye mechanism, a Velcro tape or connection mechanism, a hook-and-loop or hook-and-pile fastener or connector or touch fastener; or via bonding/gluing and/or ultrasonic connection, or via sewing or knitting, or via other suitable connecting or attachment mechanism.

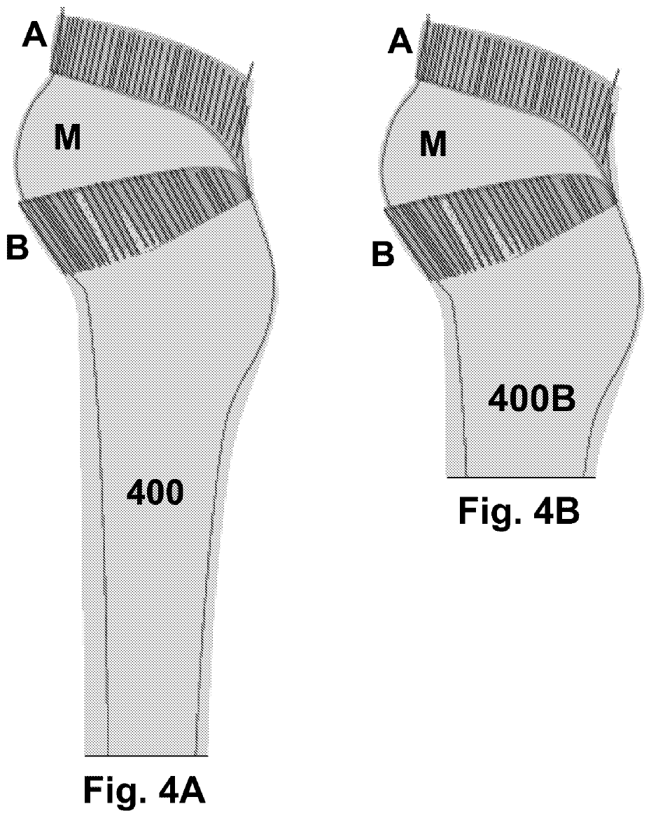

Reference is made to FIG. 4A, which is an illustration of pants/leggings 400 and an associated belly-support unit, in accordance with some demonstrative embodiments. For demonstrative purposes, pants/leggings 400 are shown as long pants/long leggings, that cover an entirety or a majority of the length of the legs, or that cover approximately ⅞ or ¾ of the length of the legs, or that cover at least ¾ or at least ⅞ of the length of the leg, or that extend to cover the legs beneath the knees of the wearer. In other embodiments, as demonstrated in FIG. 4B, the pants/leggings (denoted there 400B) may be short pants/short leggings, that cover up to 50 percent of the length of the legs, or that extend downwardly until the knees of the wearer and not downwardly below the knees of the wearer.

In a first set of implementations, pants/leggings 400 (or 400B) are integrally connected to the belly-support unit which comprises at least support belt A and support belt B. The pants/leggings 400 (or 400B) and the belly-support unit do not cover, and leave uncovered, at least a portion or region M of the pregnant belly, located between support belt A and support belt B.

In a second set of implementations, pants/leggings 400 (or 400B) are detachably-attached to the belly-support unit which comprises at least support belt A and support belt B. The pants/leggings 400 (or 400B) and the belly-support unit do not cover, and leave uncovered, at least a portion or region M of the pregnant belly, located between support belt A and support belt B.

In a third set of implementations, pants/leggings 400 (or 400B) are not attached at all to the belly-support unit which comprises at least support belt A and support belt B. Rather, the pants/leggings are an independent garment, and the belly-support unit (with at least support belts A and B) is a stand-alone accessory, which may be worn (for example) over the pants/leggings 400 (or 400B) (e.g., as an outer clothing-layer), or beneath the pants/leggings 400 (or 400B) (e.g., as an inner clothing-layer), or as a nearby clothing layer. The pants/leggings 400 (or 400B) and the belly-support unit do not cover, and leave uncovered, at least a portion or region M of the pregnant belly, located between support belt A and support belt B.

In a fourth set of implementations, pants/leggings 400 (or 400B) are integrally connected to the belly-support unit which comprises at least support belt A and support belt B, and which also comprises a stretchable fabric-region C which is located at or over region M and which covers all (or at least part of) body-region M of the wearer.

In a fifth set of implementations, pants/leggings 400 (or 400B) are detachably-attached to the belly-support unit which comprises at least support belt A and support belt B and stretchable fabric-region C that covers body-region M.

In a sixth set of implementations, pants/leggings 400 (or 400B) are not attached at all to the belly-support unit which comprises at least support belt A and support belt B and stretchable fabric-region C that covers body-region M. Rather, the pants/leggings are an independent garment, and the belly-support unit is a stand-alone accessory, which may be worn or beneath or next-to the pants/leggings 400 (or 400B).

In some embodiments, as much as the pregnant belly may grow, the distance between support belt A and support belt B may grow respectively, in an adaptive manner that adapts the distance to the growth (and shape) of the pregnant belly. In some implementations, optionally, support belt B may move more than support belt A as the pregnancy progresses, in order to support the belly weight from beneath or from the bottom. Optionally, fabric-region C (if it is included in the belly-support unit) may stretch out between the support belts (A and B) to provide coverage and comfort as well as (optionally) additional support. The stretching of support belts A and B, as well as of fabric-region C if it is included, due to the growth of the pregnant belly, may further increase the physical support to the lower-back region of the wearer.

### Second Set of Demonstrative Embodiments

In accordance with some embodiments, the interactive/adaptive belly-support unit includes the following components: at least one (or, in some implementations: exactly one) moveable belly support belt (denoted AA, and it has either exactly one support belt similar to belt A described above; or in may have two support belts similar to belts A and B described above); and a stretchable belly-support fabric-region (denoted BB), and a lower-back support fabric-region (denoted CC).

The moveable belly support belt(s) AA, the stretchable belly-covering/belly-support fabric-region BB, and the lower-back support fabric region CC, may be formed by any suitable technology, for example, knitting, weaving, seamless knitting, or the like. Their production may include plain knitting, rib, pique, mesh, and/or other types of knitting structures or knitting techniques, single-layer, double-layer, or a combination of two or more of the above. They may be formed of cotton yarn(s), polyester yarn(s), nylon yarn(s), nylon 6 yarn(s), nylon 66 or nylon 6/6 yarn(s), modal yarn(s), synthetic yarn(s), semi-synthetic yarn(s), natural yarn(s), yarn(s) from beechwood tree or beech tree, yarn(s) made of wood pulp based cellulosic fiber (e.g., European Schneider Zelkova tree, Chinese Zelkova, Zelkova Schneideriana), rayon fiber yarn(s), viscose fiber yarn(s), Spandex or Elastane or high-elasticity yarn(s) (e.g., able to stretch to at least 3 or 4 or 5 times of its idle length or size), elastic filament yarn(s), elastic spun yarn(s), and/or a combination or mixture of two or more of the above.

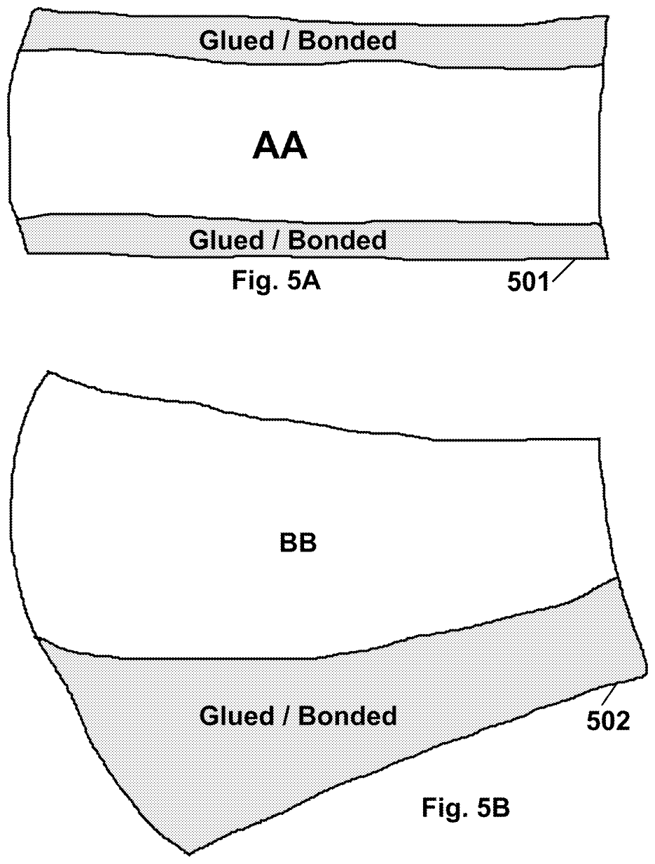

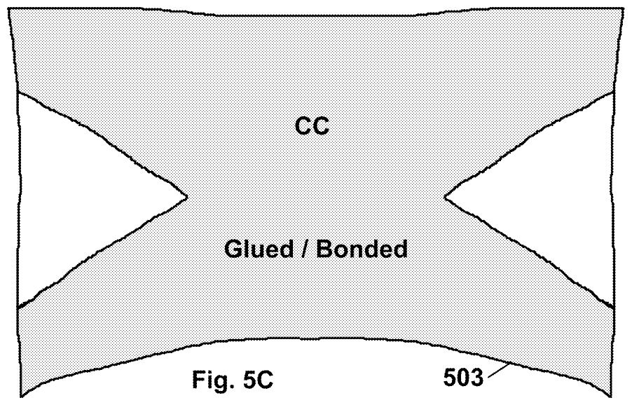

Reference is made to FIGS. 5A and 5B and 5C, which are illustrations of some possible implementations of components AA and BB and CC, respectively, in accordance with some demonstrative embodiments; demonstrating fabric-regions/fabric-elements 501 and 502 and 503, respectively.

In a demonstrative example, the moveable/convertible belly-support component AA is formed from folded of double-layer and stretchable Nylon/Spandex knitted fabric, which has been closed with stripes or strips (e.g., having stripe or strip width of 10 or 12 or 15 or 18 millimeters) of glue in between the two fabric layers. Additionally, glue stripe(s) or glue strip(s) may be added at one or more of the horizontal edges (e.g., between the two fabric layers). These two glued stripes can provide additional physical support to the belly, and may prevent rolling of the belt or the garment-region that covers at least a portion of the pregnant belly.

The stretchable belly-covering belly-support component BB may be formed of folded double-layer Nylon/Spandex fabric, which may be connected to the other garment-regions by sewing or knitting or gluing/bonding or other connection mechanism.

The lower-half of component BB can be glued in between the two fabric layers, to reduce fabric stretching, and/or to provide additional support of the lower region of the belly, while the upper-half of component BB may be free to stretch for additional belly covering.

Component CC may be formed of folded double-layer Nylon/Spandex fabric, and may be connected to components AA and BB, and also to the leggings/pants (or other garment) via sewing or gluing or other connection mechanism. In component CC, between the two fabric layers, glue can be added at a generally X-shaped gluing region, to provide additional lower-back support by component CC.

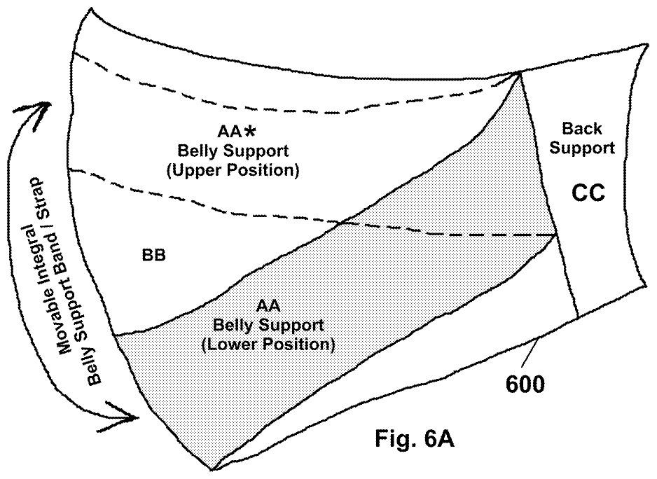

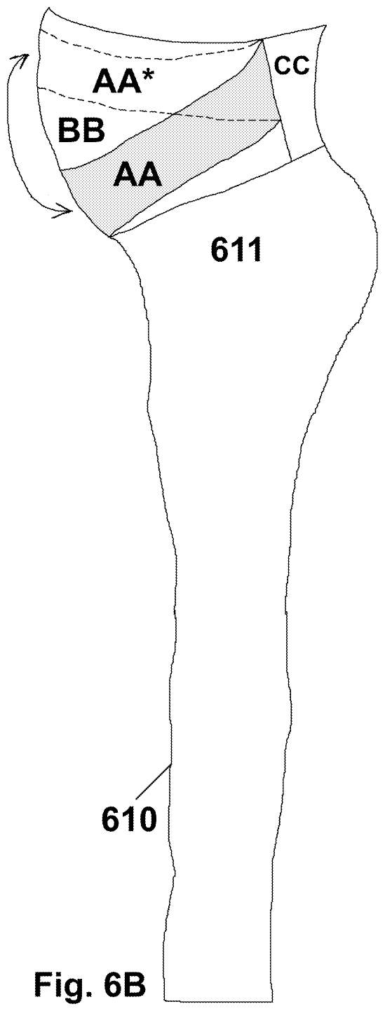

Reference is made to FIG. 6A, which is an illustration of a belly-support clothing article 600, structured from or including components AA and BB and CC, in accordance with some demonstrative embodiments. Article 600 may be an independent stand-alone clothing article or support article or accessory, which may be worn separately from pants or leggings or a skirt or a dress or short pants; or, Article 600 may be integrally connected or integrally knitted with pants or leggings; or, Article 600 may be glued or bonded or non-detachably connected or non-detachably attached to pants or leggings or other garment; or, Article 600 may be detachably-attached to pants or leggings or other garment via a detachable connection mechanism (e.g., Velcro, zipper, fastener, or the like).

Reference is also made to FIG. 6B, which is an illustration of a clothing article such as pants/leggings 610, which integrally comprises a knitted fabric-made belly-support region 610, including fabric-region components AA and BB and CC that are further integrally connected or glued or bonded or stitched or sewn or knitted-connected to pants/leggings region 611, in accordance with some demonstrative embodiments.

As demonstrated, in some embodiments, component AA may be selectively moved by the female wearer, from being in a lower position and acting as a “cradle strap”, to being in an upper position (shown in broken lines or dashed lines and denoted as AA or as AA*), and/or to being in other positions over the pregnant belly. This innovative ability of component AA to be selectively moved and positioned, may enable each female wearer to place it at or around the particular belly-region such that she obtains the best level of support and/or the most convenience, tailored to her particular needs and body; and may further enable the female wearer, optionally, in some implementations, to leave at least a portion of the pregnant belly uncovered by component AA and/or by the Article 600, if she chooses to do so.

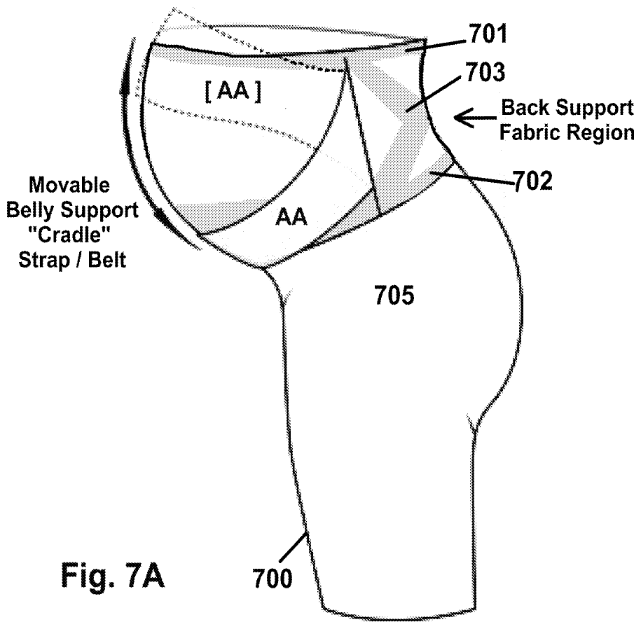

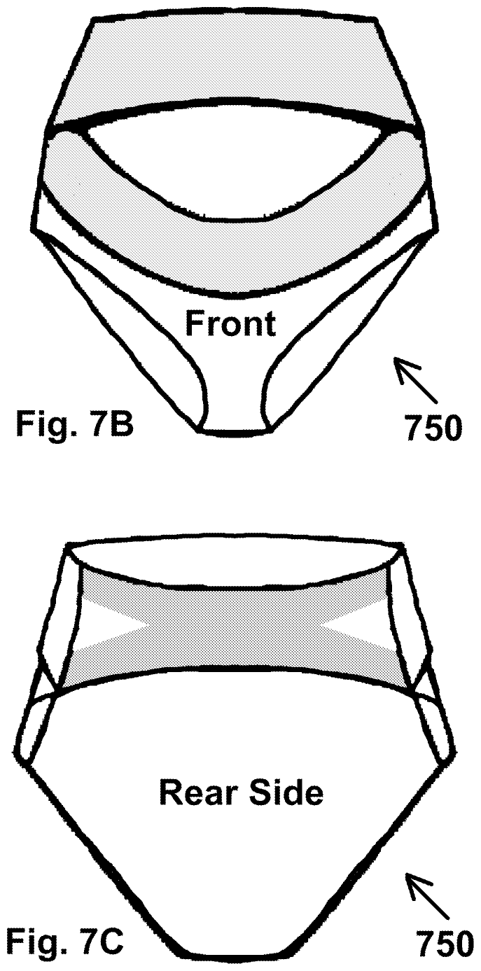

Reference is made to FIG. 7A, which is an illustration of a belly-support clothing article 700, shown schematically as being worn by a pregnant female, in accordance with some demonstrative embodiments. Clothing article 700 is demonstrated as short pants/short leggings, or as half-length pants/half-length leggings, or as knee-length pants/knee-length leggings; however, some embodiments may similarly provide a full-length pants/leggings or other suitable length (e.g., covering 7/8 or 3/4 or other ratio of the length of the legs).

Region 705 covers and encircles the buttocks, genitalia, and waist (or at least the lower portion of the waist) of the wearer; and extends downwardly along two leg-sleeves to cover at least a portion of the hips or thighs, and optionally the legs, of the human wearer.

Region 705 is integrally and continuously connected (e.g., by gluing, bonding, continuous knitting, sewing, stitching, ultrasonic fusion, taping, or the like) to a back-support fabric region, and to a movable belly-support “cradle” style fabric region (e.g., strap or belt) which can be moved from a lower position (denoted as AA in the drawing) to an upper position (dented as [AA] in the drawing) and can also be rested or kept at an interim position in between those upper and lower positions. The back-support fabric region may include, for example: an upper-side encircling strap 701, a lower-side encircling strap 702, and a rear-side (e.g., shown as an X shaped region) fabric-region; and these components provide additional support to the pregnant belly, from below it (e.g., covering at least a portion of a lower area of the pregnant belly) and from above it (e.g., covering at least a portion of an upper area of the pregnant belly) and from the rear (e.g., providing a local support to the rear side, and/or providing an elastic force that provides at least some balance to the back side or the rear side of the human wearer or to the lower-back of the human wearer, to at least partially balance the additional weight and/or size of the pregnant belly).

Reference is made to FIG. 7B, which is an illustration of a front-side of a portion 750 of a clothing article, in accordance with some demonstrative embodiments; and to FIG. 7C, which is an illustration of a rear-side of a portion 750 of a clothing article, in accordance with some demonstrative embodiments. The depicted portion 750 may be part an integral part of a clothing article such as pants/leggings/short pants, or may extend upwardly from such pants/leggings/short pants; or, in some implementations, may be a stand-alone clothing article that is particularly suitable for wearing by a pregnant female. The front-side shows the movable “cradle type” belly-support strap or belt, which is elastic and can be moved among two or more positions (two of which are shown in FIG. 7B). The rear-side shows the support region, particularly an X-shaped fabric region with increased elasticity (e.g., relative to other regions of the clothing article); the two top-side points of that X-shape fabric region may continue to a top-side elastic band; the two low-side points of that X-shape fabric region may continue to a low-side elastic band; the X-shape itself provides additional tension and elastic support; and the entire structure provides an improved support for the lower-back, and/or provides at least partial assistance to balance the weight of the pregnant belly.

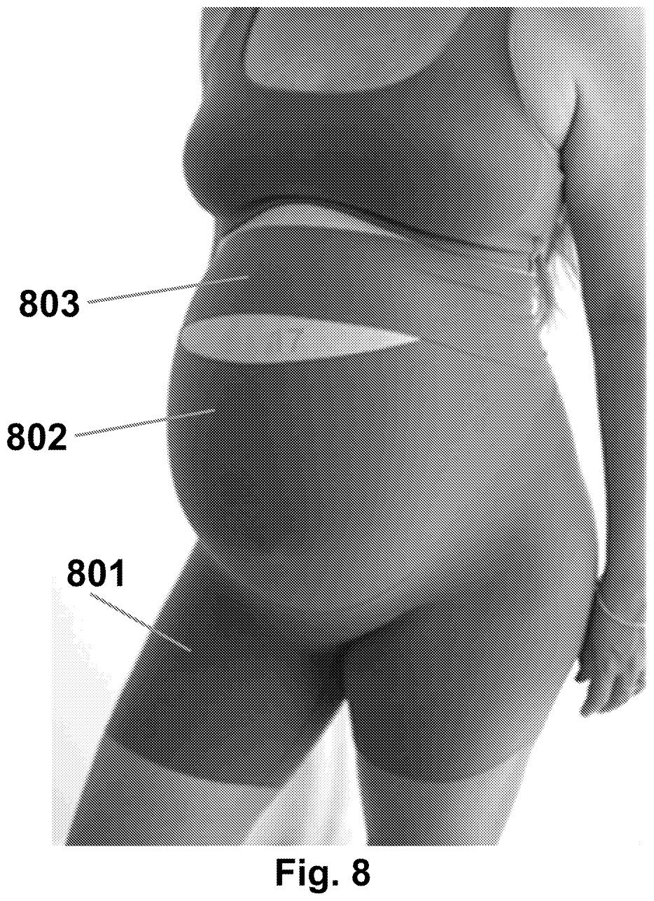

Reference is made to FIG. 8, which is an illustration of maternity pants/leggings 801 (demonstrated as short pants/short leggings), having an integrated belly-support region 802 that covers at least a portion of the pregnant belly, and also an integrated and movable belly-support strap 803 that can be moved by the human wearer to provide cover and/or support to a particular region of the pregnant belly, in accordance with some demonstrative embodiments.

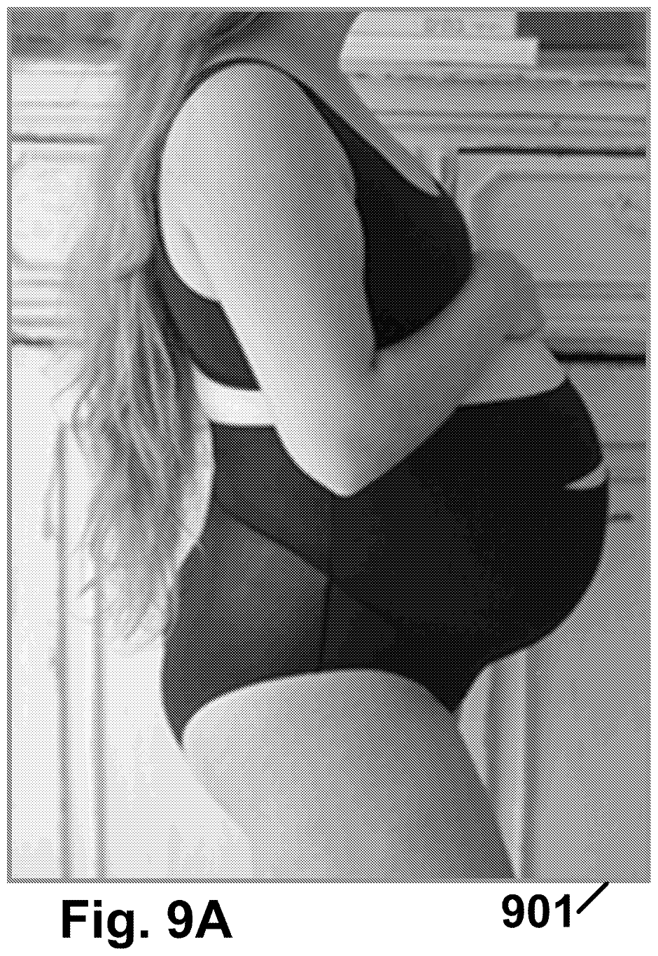

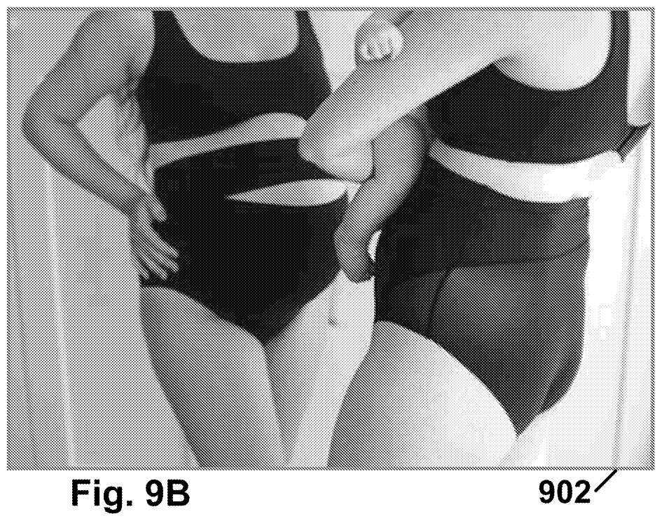

Reference is made to FIG. 9A, which is an illustration 901 demonstrating an implementation of a clothing article that provides adjustable/movable front-side belly-support, as well as back-side (e.g., lower back) support, in accordance with some demonstrative embodiments. The clothing article is demonstrated as a stand-alone underwear garment, or as stand-alone shorts (short pants/short leggings) garment; however, it can also be implemented as an integral and continuous part of long pants/long leggings.

Reference is made to FIG. 9B, which is an illustration 902 demonstrating an implementation of a clothing article that provides adjustable/movable front-side belly-support, as well as back-side (e.g., lower back) support, in accordance with some demonstrative embodiments. The clothing article is demonstrated as a stand-alone underwear garment, or as stand-alone shorts (short pants/short leggings) garment; however, it can also be implemented as an integral and continuous part of long pants/long leggings.

In some embodiments, the accessories and/or components and/or clothing articles that are shown in any of the drawings and/or that are describe above or herein, may optionally be worn by persons who are not necessarily pregnant; for example, by a female in post-pregnancy (e.g., days or weeks or months after pregnancy ended), or by a person having a large-volume belly or an oversized belly, or the like.

In accordance with some embodiments, the belly-support components are an integral and non-detachable part of the pants or leggings; and they are not an additional accessory or a separate add-on item. Accordingly, some embodiments provide leggings or pants, which may be short or long or mid-size, which integrally comprise such belly-support components, that the human wearer can selectively move or re-position or re-orient, or such that the human wearer can selectively change the angle or the orientation of one or more belts or bands of components that integrally provide belly-support. In some embodiments, the belly-support components are integrally and non-detachably connected, and non-removably connected, to an upper region of the leggings or pants; for example, via seams, via a seamless connection, via stitches, via a stitch-less connection, by gluing or bonding, by ultrasonic fusion, by knitting, by sewing, and/or other connection means. In some embodiments, the belly-support components can be worn as a first clothing layer on the body; or as a second clothing wearer (e.g., over underwear or underpants); and need not be worn as an external accessory over pants or over leggings.

In some embodiments, the elastic band(s) that form the belly-support components of the leggings/pants, may have differential elasticity; or may have regions having different levels of elasticity; in order to provide support at the back region of the wearer, while also enabling the wearer to selectively move or re-position or re-orient the belly-support components, upwardly or downwardly. In some embodiments, an elastic band that is part of the belly-support components of the leggings/pants, may have a first, reduced, elasticity level at the back side which supports the lower back region, since that particular region does not need to be selectively moved by the human wearer; and that same elastic band may have a second, increased, elasticity level at the front side (the belly side) which supports the belly, since that particular region needs to be selectively moved or re-oriented or re-positioned by the human wearer.

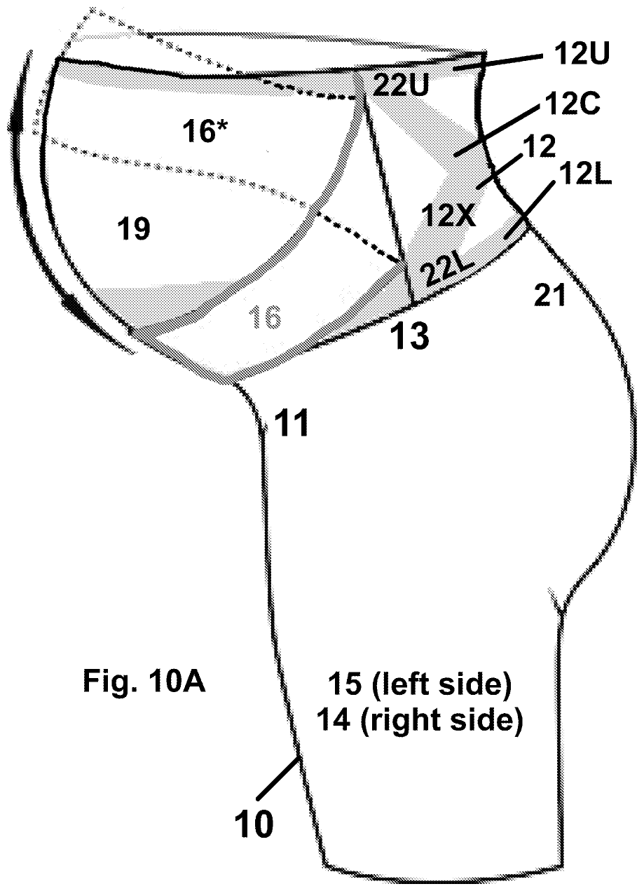

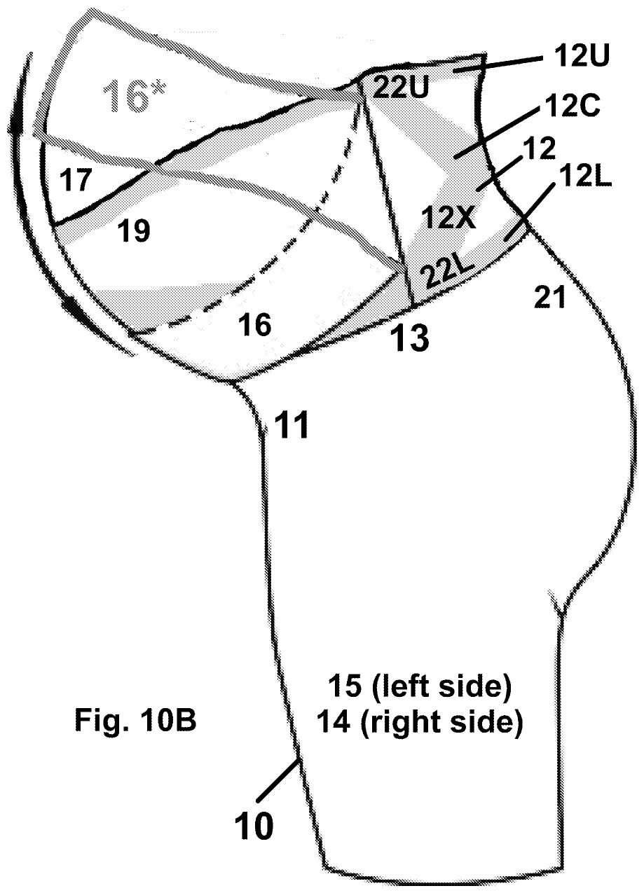

Reference is made to FIGS. 10A and 10B, which are illustrations of a clothing article having an adjustable and movable front-side belly-support strap, as well as back-side (e.g., lower back) support, in accordance with some demonstrative embodiments. In FIG. 10A, the adjustable and movable front-side belly-support strap is at its lower or lowest angular position, and provides support or additional support to the lower region (e.g., lower one-third) of the pregnant belly. In FIG. 10B, the adjustable and movable front-side belly-support strap is at its upper or higher or highest angular position, and provides support or additional support to the upper region (e.g., upper one-third or upper one-half) of the pregnant belly, while also providing or leaving a front-side uncovered aperture 17 to enable a portion of the pregnant belly to be seen and/or to get fresh air and/or to enable some direct ventilation of a central portion of the pregnant belly (e.g., for added convenience of the female wearer).

As demonstrated in FIGS. 10A and 10B and/or in other drawings, some embodiments provide a clothing article (10) for a pregnant female, the clothing article comprising: a front-side fabric-region (11), configured to cover at least genitalia of the pregnant female; a rear-side fabric-region (21), configured to cover the buttocks of the pregnant female; a waistband (13) connecting the front-side fabric-region and the rear-side fabric region, configured to encircle the waist of the pregnant female; a right-leg fabric region (14), extending downwardly from the waistband, configured to cover at least a portion of a right leg or a right hip or a right thigh of the pregnant female; a left-leg fabric region (15), extending downwardly from the waistband, configured to cover at least a portion of a left leg or a left hip or a left thigh of the pregnant female; a lower-back covering fabric region (12), extending upwardly from a rear side of the waistband, and configured to cover a lower back of the pregnant female; a movable belly-support fabric-made elastic strap (16), that is integrally and non-detachably connected to the lower-back covering fabric region (12); wherein the movable belly-support fabric-made elastic strap (16) is movable via angular pivoting between at least: (I) a lower position, in which the movable belly-support fabric-made elastic strap (16) covers at least a portion of a lower-third of a pregnant belly of the pregnant female, and (ii) an upper position, in which the movable belly-support fabric-made elastic strap (16) covers at least a portion of an upper-third of the pregnant belly of the pregnant female.

In some embodiments, the movable belly-support fabric-made elastic strap (16) is movable and adjustable along a plurality of positions between said lower position and said upper position.

In some embodiments, when the movable belly-support fabric-made elastic strap (16) is at said upper position, the clothing article provides a front-side uncovered aperture (17) to a portion of a front-side of the pregnant belly; wherein said front-side uncovered aperture (17) is bound at its top by said movable belly-support fabric-made elastic strap (16), wherein said front-side uncovered aperture (17) is bound at its bottom by said waistband (13).

In some embodiments, (i) the waistband (13) and (ii) the belly-support fabric-made elastic strap (16) are two distinct and separate fabric elements.

In some embodiments, the belly-support fabric-made elastic strap (16) is a fabric element that is external to, and separate from, the waistband (13), and at least partially overlaps externally with the waistband (13).

In some embodiments, the waistband (13) is formed of a first set of yarns, which has a first level of elasticity and which provides to the pregnant belly a first level of support; wherein the movable belly-support fabric-made elastic strap (16) is formed of a second, different, set of yarns, which has a second, different, level of elasticity and which provides to the pregnant belly a second, different, level of support.

In some embodiments, the waistband (13) is integrally and continuously connected to a belly-support fabric-region (19), that is configured to cover at least a lower one-tenth of the pregnant belly, and that remains at all times in direct contact with the waistband (13), wherein the waistband (13) and the belly-support fabric-region (19) lack any gap or aperture between them; wherein the belly-support fabric-region (19), and the movable belly-support fabric-made elastic strap (16), are two distinct and different fabric elements.

In some embodiments, the belly-support fabric-region (19) is formed of a first set of yarns, which has a first level of elasticity and which provides to the pregnant belly a first level of support; wherein the movable belly-support fabric-made elastic strap (16) is formed of a second, different, set of yarns, which has a second, different, level of elasticity and which provides to the pregnant belly a second, different, level of support.

In some embodiments, the waistband (13) has a first level of elasticity to enable the waistband to remain encircling the waist of the pregnant female; wherein the belly-support fabric-region (19) has a second level of elasticity to enable upwardly stretching of the belly-support fabric-region (19) to cover said lower one-tenth of the pregnant belly; wherein the second level of elasticity is greater than the first level of elasticity; wherein the movable belly-support fabric-made elastic strap (16) has a third level of elasticity to enable upwardly pulling and moving of the belly-support fabric-made elastic strap (16) to cover said portion of the upper-third of the pregnant belly.

In some embodiments, wherein the lower-back covering fabric region (12) comprises: an upper sub-region (12U), a central sub-region (12C), a lower sub-region (12L); wherein the upper sub-region (12U) is integrally connected to an upper elastic band (22U), that is configured to encircle an upper region of the pregnant belly; wherein the lower sub-region (12L) is integrally connected to a lower elastic band (22L), that is configured to encircle a lower region of the pregnant belly; wherein the upper elastic band (22U) and the lower elastic band (22L) are generally parallel to each other, and are vertically spaced apart from each other.

In some embodiments, the central sub-region (12C) comprises an elastic X-shaped fabric component (12X), that connects the upper elastic band (22U) and the lower elastic band (22L). In some embodiments, the upper sub-region (12U) and the lower sub-region (12L) of the lower-back covering fabric region (12) have a first level of elasticity; the central sub-region (12C) of the lower-back covering fabric region (12) has a second, different, level of elasticity.

In some embodiments, the clothing article is long pants that cover more than half of leg-length of the pregnant female. In some embodiments, the clothing article is long leggings that cover more than half of leg-length of the pregnant female. In some embodiments, the clothing article is short pants that cover not more than half of leg-length of the pregnant female. the clothing article is short leggings that cover not more than half of leg-length of the pregnant female. In some embodiments, the clothing article is an undergarment or underwear.

In some embodiments, optionally, the combined/hybrid garment may comprise pants (or shorts, or short pants, or long pants, or leggings) that have an integral and integrated, selectively adjustable, location-modifiable, belly-support region; which can accompany a female wearer throughout her entire pregnancy, starting from pre-pregnancy or early-pregnancy time, through pregnancy stages, and optionally post-delivery time; since the elasticity and/or structure and/or location and/or configuration of the adjustable, location-modifiable, belly-support region allows the wearer to selectively adjust and modify the location and/or the angular location and/or the slanting of the belly-support region or band or belt, for supporting the belly as it grows during pregnancy and/or as it shrinks (abruptly and/or gradually) post-delivery; without requiring the female wearer to manually connect and/or disconnect a separate accessory or a separate belt or band; and while providing an innovative singular, unified, hybrid or combine garment that integrally comprises the adjustable belly-support region.

Some embodiments may be implemented by using a machine or an automated or semi-automatic production line, which may comprise, for example: cutting unit, welding unit, bonding unit, sewing unit, ultrasonic operations unit, ultrasonic cutting unit, gluing unit, ultrasonic bonding unit, ultrasonic gluing unit, folding unit, connection-creating unit, conveyor belt, robotic arm, control unit, workstation; as well as suitable hardware components and/or software components, for example, processor to execute code, memory unit, storage unit, input unit (keyboard, mouse, touch-screen), output unit (screen, touch-screen), modems, transceivers or transmitters or receivers, wireless and/or wired communication links and/or transceivers or transmitters or receivers, power sources, Operating System (OS) and suitable applications, or the like.

Functions, operations, components and/or features described herein with reference to one or more embodiments, may be combined with, or may be utilized in combination with, one or more other functions, operations, components and/or features described herein with reference to one or more other embodiments, or vice versa.

While certain features of some embodiments have been illustrated and described herein, many modifications, substitutions, changes, and equivalents may occur to those skilled in the art. Accordingly, the claims are intended to cover all such modifications, substitutions, changes, and equivalents.

1. A clothing article (10) for a pregnant female, the clothing article comprising:
a front-side fabric-region (11), configured to cover at least genitalia of the pregnant female;
a rear-side fabric-region (21), configured to cover the buttocks of the pregnant female;
a waistband (13) connecting the front-side fabric-region and the rear-side fabric region, configured to encircle the waist of the pregnant female;
a right-leg fabric region (14), extending downwardly from the waistband, configured to cover at least a portion of a right leg or a right hip or a right thigh of the pregnant female;
a left-leg fabric region (15), extending downwardly from the waistband, configured to cover at least a portion of a left leg or a left hip or a left thigh of the pregnant female;
a lower-back covering fabric region (12), extending upwardly from a rear side of the waistband, and configured to cover a lower back of the pregnant female;
a movable belly-support fabric-made elastic strap (16), that is integrally and non-detachably connected to the lower-back covering fabric region (12);
wherein the movable belly-support fabric-made elastic strap (16) is movable via angular pivoting between at least: (I) a lower position, in which the movable belly-support fabric-made elastic strap (16) covers at least a portion of a lower-third of a pregnant belly of the pregnant female, and (ii) an upper position, in which the movable belly-support fabric-made elastic strap (16) covers at least a portion of an upper-third of the pregnant belly of the pregnant female.

2. The clothing article according to claim 1,
wherein the movable belly-support fabric-made elastic strap (16) is movable and adjustable along a plurality of positions between said lower position and said upper position.

3. The clothing article according to claim 2,
wherein, when the movable belly-support fabric-made elastic strap (16) is at said upper position, the clothing article provides a front-side uncovered aperture (17) to a portion of a front-side of the pregnant belly,
wherein said front-side uncovered aperture (17) is bound at its top by said movable belly-support fabric-made elastic strap (16),
wherein said front-side uncovered aperture (17) is bound at its bottom by said waistband (13).

4. The clothing article according to claim 3,
wherein (i) the waistband (13) and (ii) the belly-support fabric-made elastic strap (16) are two distinct and separate fabric elements.

5. The clothing article according to claim 3,
wherein the belly-support fabric-made elastic strap (16) is a fabric element that is external to, and separate from, the waistband (13), and at least partially overlaps externally with the waistband (13).

6. The clothing article according to claim 3,
wherein the waistband (13) is formed of a first set of yarns, which has a first level of elasticity and which provides to the pregnant belly a first level of support;
wherein the movable belly-support fabric-made elastic strap (16) is formed of a second, different, set of yarns, which has a second, different, level of elasticity and which provides to the pregnant belly a second, different, level of support.

7. The clothing article according to claim 3,
wherein the waistband (13) is integrally and continuously connected to a belly-support fabric-region (19), that is configured to cover at least a lower one-tenth of the pregnant belly, and that remains at all times in direct contact with the waistband (13), wherein the waistband (13) and the belly-support fabric-region (19) lack any gap or aperture between them;
wherein the belly-support fabric-region (19), and the movable belly-support fabric-made elastic strap (16), are two distinct and different fabric elements.

8. The clothing article according to claim 7,
wherein the belly-support fabric-region (19) is formed of a first set of yarns, which has a first level of elasticity and which provides to the pregnant belly a first level of support;
wherein the movable belly-support fabric-made elastic strap (16) is formed of a second, different, set of yarns, which has a second, different, level of elasticity and which provides to the pregnant belly a second, different, level of support.

9. The clothing article according to claim 7,
wherein the waistband (13) has a first level of elasticity to enable the waistband to remain encircling the waist of the pregnant female;
wherein the belly-support fabric-region (19) has a second level of elasticity to enable upwardly stretching of the belly-support fabric-region (19) to cover said lower one-tenth of the pregnant belly; wherein the second level of elasticity is greater than the first level of elasticity;
wherein the movable belly-support fabric-made elastic strap (16) has a third level of elasticity to enable upwardly pulling and moving of the belly-support fabric-made elastic strap (16) to cover said portion of the upper-third of the pregnant belly.

10. The clothing article according to claim 3,
wherein the lower-back covering fabric region (12) comprises:
an upper sub-region (12U),
a central sub-region (12C),
a lower sub-region (12L);
wherein the upper sub-region (12U) is integrally connected to an upper elastic band (22U), that is configured to encircle an upper region of the pregnant belly;
wherein the lower sub-region (12L) is integrally connected to a lower elastic band (22L), that is configured to encircle a lower region of the pregnant belly;
wherein the upper elastic band (22U) and the lower elastic band (22L) are generally parallel to each other, and are vertically spaced apart from each other.

11. The clothing article according to claim 10,
wherein the central sub-region (12C) comprises an elastic X-shaped fabric component (12X), that connects the upper elastic band (22U) and the lower elastic band (22L).

12. The clothing article according to claim 11,
wherein the upper sub-region (12U) and the lower sub-region (12L) of the lower-back covering fabric region (12) have a first level of elasticity;
wherein the central sub-region (12C) of the lower-back covering fabric region (12) has a second, different, level of elasticity.

13. The clothing article according to claim 3,
wherein the clothing article is long pants that cover more than half of leg-length of the pregnant female.

14. The clothing article according to claim 3,
wherein the clothing article is long leggings that cover more than half of leg-length of the pregnant female.

15. The clothing article according to claim 3,
wherein the clothing article is short pants that cover not more than half of leg-length of the pregnant female.

16. The clothing article according to claim 3,
wherein the clothing article is short leggings that cover not more than half of leg-length of the pregnant female.

17. The clothing article according to claim 3,
wherein the clothing article is an undergarment or underwear.
